(19) United States
(12) Patent Application Publication
(10) Pub. No.: US 2010/0151490 A1
(43) Pub. Date: Jun. 17, 2010

(54) Antigenic Protein Conjugates and Process for Preparing Same

(75) Inventors: Stuart J. Blincko, Northumberland Heath (GB); Deborah A. Blackwell, Tonbridge Kent (GB); Emma J. Doran, Gravesend Kent (GB); Brian C. Rodgers, Bromley (GB)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/712,564

(22) Filed: Feb. 25, 2010

Related U.S. Application Data
(63) Continuation of application No. 11/845,941, filed on Aug. 28, 2007, now abandoned.
(60) Provisional application No. 60/841,801, filed on Sep. 1, 2006.

(51) Int. Cl. G01N 33/53; C07K 1/00; C12N 9/00; C12N 9/08

(52) U.S. Cl. 435/7.1; 530/403; 435/183; 435/192

(57) ABSTRACT

An improved process for the preparation of antigenic protein conjugates is provided. The conjugates preferably are formed through reaction with one or more free sulfhydryl groups in the antigenic protein. The process of the present invention preferably employs a trialkylphosphine as the reducing agent and allows for reduction of disulfide bonds in the antigenic protein and conjugation with a conjugate moiety, preferably in a single reaction vessel (i.e. “in situ”) because the process optimally does not require the removal of the reducing agent before subsequent addition of the sulfhydryl reactive agent. Antigenic protein conjugates prepared by the in situ process and their use in diagnostic immunoassays are also provided.

### RELATED APPLICATION INFORMATION

This application is a continuation of U.S. patent application Ser. No. 11/845,941, filed on Aug. 28, 2007, which claims the benefit of U.S. Provisional Patent Application No. 60/841,801 filed on Sep. 1, 2006, each of which is hereby incorporated by reference in its entirety.

### FIELD OF THE INVENTION

Among other things, the present invention relates to the field of protein conjugates and, in particular, to a process for preparing a conjugate of an antigenic protein.

### BACKGROUND OF THE INVENTION

Protein conjugates, in which a label, tag or other moiety are attached to a protein, can be formed by a number of methods known in the art. Due to the low number of thiol (sulfhydryl) groups present in proteins as compared to other reactive groups, such as amines, the use of thiol groups to conjugate labels or tags to proteins allows for a more focused approach that is less likely to block or inactivate protein activity.

Sulfhydryl groups are present in proteins in the form of cysteine residues, and are frequently present in the form of disulfide bonds. Prior to conjugation, therefore, the protein must be treated with a suitable reducing agent in order to expose the sulfhydryl groups. Reducing agents that are typically used to reduce protein disulfide bonds include dithiothreitol (DTT) and β-mercaptoethanol (β-ME). The use of either of these compounds as a reducing agent requires the subsequent step of removing the reducing agent before introduction of a sulfhydryl reactive reagent as the thiol functionality of the reducing agent can also react with the sulfhydryl reactive agent, thereby inhibiting and reducing the conjugation efficiency. The use of thiol-containing reducing agents, therefore, necessitates additional purification steps leading to increased processing time with the concomitant increased likelihood that the sulfhydryl groups will oxidize and reform intermolecular and intramolecular disulfide bonds.

The water soluble tris(carboxyethyl)phosphine (TCEP) is also used to reduce disulfide bonds (J. Org. Chem., 56, 2648-2650 (1991)). TCEP provides the advantage of being relatively odorless compared to DTT and 13-ME. TCEP is also kinetically stable in aqueous solution, and retains its reductive effectiveness across a wide pH range. TCEP has been shown to be significantly more stable than DTT at pH values above 7.5, and is a faster and stronger reductant than DTT at pH values below 8.0.

While TCEP has been generally held to be selective for the reduction of disulfide bonds, and unreactive or minimally reactive toward many other functionalities, some recent studies have indicated that TCEP may in fact react with certain thiol-reactive functionalities. In comparing properties of DTT and TCEP, researchers (Getz, E. B., et al., Anal. Biochemistry, 273, 73-80 (1999)) reported that both DTT and TCEP inhibit the labeling of sulfhydryl groups with maleimide when compared to labeling with no reductant present, although this inhibition is more pronounced with DTT. When labeling with iodoacetamide, it was found that, at low concentrations (i.e., 0.1 mM), the presence of either TCEP or DTT has little effect on labeling efficiency. However, at higher concentrations (i.e., 1.0 mM), TCEP is more deleterious to iodoacetamide attachment than a corresponding amount of DTT. Similarly, other researchers (Shafer, D. E., et al., Anal. Biochemistry, 282, 161-164 (2000)) have reported that TCEP combines rapidly with both iodoacetamide and maleimide groups, making it necessary to remove excess TCEP from the thiol-peptide solution before the conjugation step.

TCEP reduction of disulfide bonds in proteins has been employed in a number of contexts. For example, for partial reduction of proteins in preparation for matrix assisted laser desorption/ionization and liquid secondary ionization mass-spectrometry (Fischer, W. H., et al., Rapid Commun. Mass. Spectrom., 7, 225-228 (1993), for fluorescence labeling of proteins for fluorescence polarization or FRET studies (Bergendahl, V., et al., Anal. Biochemistry, 307, 368-374 (2002)), and for limited reduction of protein disulfide bonds for mapping purposes (Wu, J. and Watson, J. T., Protein Sci., 6, 391-398 (1997)).

This background information is provided for the purpose of making known information believed by the applicant to be of possible relevance to the present invention. No admission is necessarily intended, nor should be construed, that any of the preceding information constitutes prior art against the present invention.

All patents and publications referred to herein are hereby incorporated in their entirety by reference.

### SUMMARY OF THE INVENTION

An object of the present invention is to provide a process for the preparation of antigenic protein conjugates. In accordance with one embodiment of the present invention, there preferably is provided a process for preparing an antigenic protein conjugate that comprises the steps of: (a) contacting an antigenic protein comprising one or more disulfide bonds with a trialkylphosphine under conditions that allow reduction of at least one of the one or more disulfide bonds to provide a reduced antigenic protein comprising at least a pair of sulfhydryl residues, and (b) contacting the reduced antigenic protein with a sulfhydryl reactive reagent comprising a conjugate moiety and a thiol-reactive functionality under conditions that allow reaction of at least one of the sulfhydryl residues with the sulfhydryl reactive reagent to form said antigenic protein conjugate.

In accordance with another aspect of the present invention, there preferably is provided an antigenic protein conjugate prepared by a process comprising the steps of: (a) contacting an antigenic protein comprising one or more disulfide bonds with a trialkylphosphine under conditions that allow reduction of at least one of the one or more disulfide bonds to provide a reduced antigenic protein comprising at least a pair of sulfhydryl residues, and (b) contacting the reduced antigenic protein with a sulfhydryl reactive reagent comprising a conjugate moiety and a thiol-reactive functionality under conditions that allow reaction of at least one of the sulfhydryl residues with the sulfhydryl reactive reagent to form the antigenic protein conjugate.

In accordance with another aspect of the present invention, there preferably is provided a method for detecting antibodies to an antigenic protein in a sample comprising: (a) contacting the sample with an antigenic protein conjugate under conditions that allow the formation of an antibody:antigenic protein conjugate complex, wherein the antigenic protein conjugate comprises the antigenic protein conjugated to a detectable label and preferably is prepared by a process comprising the steps of: (i) contacting the antigenic protein with a trialkylphosphine under conditions that allow reduction of one or more disulfide bonds in the antigenic protein to provide a reduced antigenic protein comprising at least a pair of sulfhydryl residues, and (ii) contacting the reduced antigenic protein with a sulfhydryl reactive reagent comprising a detectable label and a thiol-reactive functionality under conditions that allow reaction of at least one of said sulfhydryl residues with the sulfhydryl reactive reagent to form the antigenic protein conjugate, and (b) detecting any antibody:antigenic protein conjugate complexes formed in step (a).

### BRIEF DESCRIPTION OF THE DRAWINGS

These and other features of the invention will become more apparent in the following detailed description in which reference is made to the appended drawings.

FIG. 1 presents a schematic diagram of the reduction and conjugation steps employed for preparing a horseradish peroxidase (HRP)-conjugated protein by the in situ process according to an illustrative embodiment of the present invention. This same process can be applied for recombinant, synthetic or isolated antigenic proteins.

FIG. 2 is a bar chart that presents the results of a comparison of the sensitivity of replicate samples of horseradish peroxidase (HRP)-NS3 protein conjugates (solid bars) prepared by in situ and conventional techniques using serum as a negative control (open bars) for the test conditions: (A) 1 in 800 dilution, in situ preparation, without DTT or TCEP; (B) 1 in 1600 dilution, in situ preparation, without DTT or TCEP; (C) 1 in 200 dilution, conventional preparation, without DTT; (D) 1 in 200 dilution, conventional preparation, with TCEP; (E) 1 in 200 dilution, conventional preparation, without DTT or TCEP.

FIG. 3 is a bar chart that presents the ability of various horseradish peroxidase (HRP)-gp41-p24 recombinant protein (dx589) conjugates prepared by in situ techniques employing 5 molar equivalents of HRP-maleimide and varying concentrations of tris(carboxyethyl)phosphine (TCEP) to bind to anti-HIV antibodies present in HIV negative samples (open bars) and HIV positive serum samples (solid bars—first bar, QC1362; second bar, QC1363) where the following molar equivalents of TCEP were used during preparation: (A) 0; (B) 2.5; (C) 5; (D) 10; (E) 20; (F) 40; and (G) Reference (preparation by the conventional technique).

FIG. 4 is a bar chart that presents the ability of various horseradish peroxidase (HRP)-gp41-p24 recombinant protein (dx589) conjugates prepared by in situ techniques employing varying concentrations of HRP-maleimide and 5 molar equivalents of tris(carboxyethyl)phosphine (TCEP) to bind to anti-HIV antibodies present in HIV negative samples (open bars) and HIV positive serum samples (solid bars—first bar, QC1362; second bar, QC1363) where the following molar equivalents of HRP-maleimide were used during preparation: (A) 2.5; (B)₅; (C) 10; (D) 20; and (E) Reference (preparation by the conventional technique).

FIG. 5 is a bar chart that presents the results of a titration study to determine the ability of a horseradish peroxidase (HRP)-gp41-p24 recombinant protein (dx589) conjugate prepared by in situ techniques to bind to anti-HIV antibodies HIV negative samples (open bars) and HIV positive serum samples (solid bars—first bar, QC1362; second bar, QC1363) where the following dilutions of the protein conjugate were tested: (A) 1 in 13,000; (B) 1 in 26,000; and (C) 1 in 52,000.

FIG. 6 is a scatter plot that presents the results of a comparison of the specificity of horseradish peroxidase (HRP)-gp41-p24 recombinant protein (dx589) conjugates prepared by the in situ process (X-axis) and the conventional technique (Y-axis). The in situ conjugate prepared without SDS is shown with open triangles and the in situ conjugate prepared with SDS is shown with solid diamonds.

### DETAILED DESCRIPTION OF THE INVENTION

The present invention provides among other things an improved process for the preparation of antigenic protein conjugates. The conjugates preferably are formed through reaction with one or more free sulfhydryl groups in the antigenic protein. The process of the present invention preferably employs a trialkylphosphine as the reducing agent. Further, the process preferably allows for reduction of disulfide bonds in the antigenic protein and conjugation with a conjugate moiety in a single reaction vessel (i.e. “in situ”) as the process does not require the removal of the reducing agent before subsequent addition of the sulfhydryl reactive agent.

In accordance with one aspect of the present invention, the antigenic protein conjugate provided by the process of the present invention preferably demonstrates enhanced antibody-binding properties when compared to the corresponding conjugate prepared by conventional methods, such as those employing DTT, and thus represents an improvement over currently available sulfhydryl derivatized protein conjugates. The present invention thus further preferably provides for improved antigenic protein conjugates prepared by the in situ process. Without being limited to any particular theory or mechanism, it is proposed that the use of a trialkylphosphine reducing agent in situ with a sulfhydryl reactive agent comprising the conjugate moiety to be conjugated to the protein allows the protein to be held in an open conformation, thereby preventing protein folding that may mask or conceal epitopes. Regardless of mechanism, it is a novel and surprising result that a key epitope containing a cysteine is not masked, but rather is held open, by the process of the invention

In one embodiment of the present invention, preferably conjugation of the conjugate moiety with the free sulfhydryl groups of the antigenic protein in the presence of the trialkylphosphine reducing agent minimizes the reformation of intermolecular or intramolecular disulfide bonds that may otherwise take place prior to the conjugation step. In accordance with this embodiment and in contrast to protein conjugates prepared by conventional techniques, preferably the antigenic protein conjugate prepared by the in situ process can be subsequently employed to efficiently bind to a cognate antibody in the absence of additional reducing agent.

In accordance with another aspect of the present invention, the in situ process preferably provides for incorporation of sufficient conjugate moiety into the antigenic protein without the requirement for additional derivatizing agents, such as N-succinimidyl-5-acetylthioacetate (SATA).

In a further aspect, preferably the process of the present invention represents a simplified conjugation procedure relative to conventional methods by minimizing the number of steps required to prepare the protein conjugate. In one embodiment, the elimination of a purification step to remove the reducing agent preferably decreases the overall time for the process relative to conventional conjugation methods. The decreased time required for the process of the present invention also minimizes the exposure of the reactants to an oxidizing environment, thereby decreasing the likelihood that the sulfhydryl groups will oxidize and reform intermolecular and intramolecular disulfide bonds.

In another aspect, preferably the process of the present invention is readily reproducible providing a more consistent product than conventional methods.

The protein conjugates provided by the process of the present invention have application in a number of contexts where an enhanced interaction between the protein and antibodies that bind the protein and/or consistency of product is advantageous. Examples include, but are not limited to, detection, purification and assay development applications. In a further aspect, the present invention thus provides for a method of detecting antibodies to an antigenic protein that employs an antigenic protein conjugate prepared by the in situ process.

### DEFINITIONS

Unless defined otherwise, all technical and scientific terms used herein have the same meaning as commonly understood by one of ordinary skill in the art to which this invention belongs.

The term “cycloalkyl” refers to a cyclic or polycyclic alkyl group containing 3 to 15 carbon atoms (unless otherwise specified). For polycyclic groups, these may be multiple condensed rings in which one of the distal rings may be aromatic (e.g. tetrahydronaphthalene, and the like).

The term “aryl” refers to an aromatic carbocyclic group having at least one aromatic ring (e.g., phenyl or biphenyl) or multiple condensed rings in which at least one ring is aromatic, (e.g., 1,2,3,4-tetrahydronaphthyl, naphthyl, anthryl, phenanthryl, 9-fluorenyl, and the like).

The terms “heterocycle” or “heterocyclic group” refer to a saturated, unsaturated, or aromatic carbocyclic group having a single ring (e.g., morpholino, pyridyl or furyl) or multiple condensed rings (e.g., naphthpyridyl, quinoxalyl, quinolinyl, indolizinyl, indanyl or benzo[b]thienyl) and having at least one hetero atom, such as N, O or S, within the ring.

The term “heteroaryl” refers to a heterocycle in which at least one heterocyclic ring is aromatic.

As used herein, the term “about” refers to approximately a +/−10% variation from the stated value. It is to be understood that such a variation is always included in any given value provided herein, whether or not it is specifically referred to.

### Process for the Preparation of Antigenic Protein Conjugates

The present invention preferably provides an in situ process for preparing a sulfhydryl derivatized antigenic protein conjugate. The process preferably comprises a reduction step in which the disulfide bond(s) of the protein are reduced with a trialkylphosphine to produce free sulfhydryl residues, and a conjugation step which comprises reaction of the free sulfhydryl residues with a sulfhydryl reactive reagent comprising a conjugate moiety to form the sulfhydryl derivatized antigenic protein conjugate. In accordance with the present invention, preferably the trialkylphosphine does not need to be removed from the reaction mixture prior to the conjugation step.

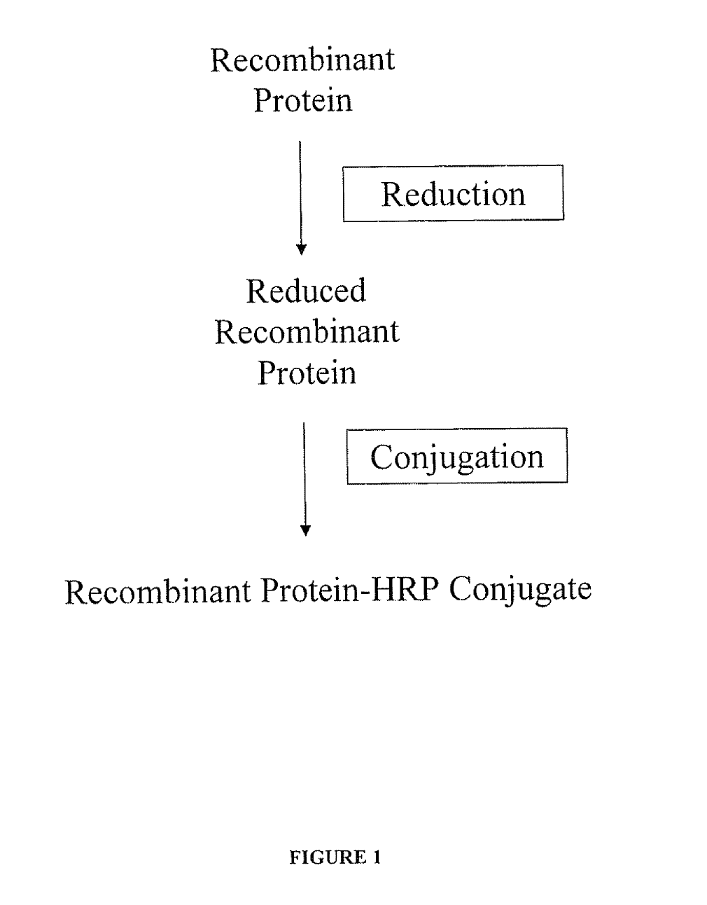

In one embodiment, the in situ process preferably comprises the reduction and conjugation steps shown generally in FIG. 1 for a recombinant protein (also applicable to isolated and/or synthetic antigenic proteins). In accordance with this embodiment, preferably the process can be completed in as little as 3 hours, for example, when the reduction step is conducted at 37° C.

Other components may optionally be included as part of the reduction and/or the conjugation step in order to facilitate reduction of the disulfude bonds or conjugation of the conjugate moiety, respectively.

The process may further comprise optional additional steps subsequent to the conjugation step, if desired, in order to optimize the protein conjugate for downstream applications. For example, the process can include a blocking step to block any unreacted free sulfhydryl groups remaining after conjugation, one or more purification steps, and the like. This is all set forth in more detail below (e.g. description of Reaction Conditions), and also in the Examples which follow.

### Antigenic Proteins

The in situ process of the present invention preferably can be applied to a variety of antigenic proteins for which detection of antibodies against the protein is desirable, provided that the protein contains at least one disulfide bond. The disulfide bond can be present as an intramolecular disulfide bond (i.e., within the protein molecule), or as an intermolecular disulfide bond (i.e., between two protein molecules, for example between two molecules of a dimeric protein). In one embodiment, the in situ process is applied to an antigenic protein that comprises an intramolecular disulfide bond. In another embodiment, the in situ process is applied to an antigenic protein that comprises an intermolecular disulfide bond.

Examples of suitable antigenic proteins for use in the process of the present invention include, but are not limited to, proteins that indicate the presence of a disease or an infection, such as, for example, viral proteins, bacterial proteins, fungal proteins, proteins that trigger autoimmune responses, and the like. Non-limiting examples include structural and non-structural proteins of human immunodeficiency virus (HIV), hepatitis C virus (HCV), hepatitis B virus (HBV), hepatitis A virus, West Nile virus, respiratory syncytial virus, influenza virus, rabies virus, human papilloma virus (HPV), Epstein Barr virus (EBV), polyoma virus, SARS coronavirus, avian influenza virus; secreted proteins from E. coli, Shigella spp., Chlamydia spp., and Pertussis spp.; and human proteins such as human chorionic gonadotrophin (hCG), luteinizing hormone (LH) and thyroid stimulating hormone (TSH).

In one embodiment of the present invention, preferably the in situ process is applied to a viral protein. In another embodiment, the in situ process is applied to a HCV or HIV protein. In another embodiment, preferably the process is applied to a HCV protein.

The protein employed in the in situ process preferably can be an isolated protein, a recombinant protein, or a synthetic protein. Methods of isolating proteins are well known in the art, as are methods of producing recombinant proteins (see, for example, Coligan et al. (Ed.), Current Protocols in Protein Science, John Wiley & Sons, Inc. New York, N.Y.; Ausubel, et al. (Ed.), Current Protocols in Molecular Biology, John Wiley & Sons, Inc. New York, N.Y.). Alternatively, the protein preferably can be obtained from commercial sources. A wide variety of isolated or recombinant antigenic proteins are currently commercially available, for example, from Sigma-Aldrich (St. Louis, Mo.), Innogenetics N.V. (Gent, Belgium), Advanced Biotechnologies Inc. (Columbia, Md.), and Serotec (Raleigh, N.C.).

The in situ process of the present invention preferably is equally applicable to full-length proteins, truncated proteins and antigenic protein fragments that comprise one or more disulfide bonds. Such fragments preferably can be generated from an isolated or recombinant protein by art known fragmentation methods, such as protease digestion, or they can be synthesized chemically, for example by standard solid phase peptide synthesis, or prepared using recombinant techniques. Similarly, the in situ process preferably can be applied to native proteins or mutants versions thereof, provided that such mutant proteins comprise at least one disulfide bond and retain one or more epitopes of the native protein.

In one embodiment of the present invention, preferably the in situ process is applied to a full-length protein, or a substantially full-length protein.

### Trialkylphosphine Reducing Agents

Disulfide bonds of the protein preferably are reduced using a trialkylphosphine reducing agent, each reduced disulfide bond providing two sulfhydryl groups, one or both of which can be further reacted with a sulfhydryl reactive reagent to form the protein conjugate. The trialkylphosphine preferably has the general formula (I):

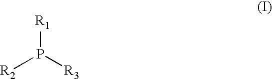

wherein: R₁, R₂ and R₃ are each independently a substituted or unsubstituted alkyl group.

A number of trialkylphosphines are known in the art and are available commercially. Examples include, but are not limited to, tris(carboxyethyl)phosphine (TCEP) (Invitrogen-Molecular Probes, Carlsbad, Calif.; Pierce Biotechnology Inc., Rockford, Ill.; Sigma-Aldrich, St. Louis, Mo.), tris(2-cyanoethyl)phosphine (Invitrogen-Molecular Probes, Carlsbad, Calif.) and tributylphosphine (Sigma-Aldrich, St. Louis, Mo.). Immobilized TCEP is also available commercially (for example, from Pierce Biotechnology Inc., Rockford, Ill.) and can be employed in the process of the present invention.

In one embodiment of the present invention, preferably the trialkylphosphine is the water-soluble tris(carboxyethyl)phosphine (TCEP). Alternatively, in another embodiment, preferably the trialkylphosphine is tris(2-cyanoethyl)phosphine. Not willing to be bound by any theory, it appears that the increased hydrophobicity of tris-(2-cyanoethyl)phosphine relative to that of TCEP can allow for greater penetration of the reducing agent into the hydrophobic regions of the protein, which may yield greater reactivity with buried disulfides. In another embodiment, preferably the trialkylphosphine is tributylphosphine.

### Other Components

One or more other components that facilitate the reduction of the disulfude bonds in the protein optionally can be included in the reduction reaction. For example, in one embodiment, a denaturant or detergent can be added to the reaction mixture. Detergents/denaturants may enhance penetration of the trialkylphosphine into the hydrophobic core of the protein. Suitable denaturants or detergents that do not interfere with the ability of the trialkylphosphine to reduce the disulfide bond(s) in the protein can be readily selected by one skilled in the art. Examples include, but are not limited to, sodium dodecyl sulfate (SDS), urea, guanidine, and combinations thereof. The amount of the denaturant to be added can be determined as is known in the art. For example, the reactions can be carried out in the presence of varying amounts of detergent to determine the concentration resulting in optimum results.

In one embodiment, preferably a detergent is included in the reduction step of the process. In another embodiment, preferably SDS is included in the reduction step of the process.

Providing access to a disulfide bond located in the hydrophobic core of a protein also preferably may be facilitated by adjusting the pH of the reaction mixture to make it acidic (for example, between about pH 2.5 and about pH 5.0) or basic (for example, between about pH 9.0 and about pH 11.0). As trialkylphosphines, such as TCEP, retain their effectiveness over a wide pH range, adjustment of the pH preferably will not affect the reducing capability of the trialkylphosphine. Optionally, adjustment of the pH can be achieved through the use of various pH-modifying reagents known in the art, such as acids, bases and buffers.

In one embodiment, preferably pH-modifying reagents are included in the reduction step of the process.

### Sulfhydryl Reactive Reagent

The sulfhydryl reactive reagent employed in the process of the present invention preferably comprises a conjugate moiety linked to a thiol-reactive functionality. The conjugate moiety preferably can be linked to the thiol-reactive functionality directly through a functional group on the conjugate moiety, or indirectly, through a linker.

### Conjugate Moiety

The process of the present invention preferably can be employed to conjugate a variety of useful moieties to the antigenic protein via the free sulfhydryl groups revealed by the reduction step. Examples of conjugate moieties contemplated by the present invention include, but are not limited to, detectable labels, moieties that facilitate purification, and moieties that facilitate or provide immobilization.

In one embodiment of the present invention, preferably the conjugate moiety is a detectable label. Non-limiting examples of suitable detectable labels include those that can be directly detected such as radioisotopes, fluorophores, chemiluminophores, enzymes, colloidal particles, fluorescent microparticles, and the like. The detectable label preferably is itself detectable or optionally may be reacted with one or more additional compounds to generate a detectable product. Thus, one skilled in the art will understand that directly detectable labels may require additional components, such as substrates, triggering reagents, light, and the like to enable detection of the label. Examples of detectable labels include, but are not limited to, chromogens, radioisotopes (such as for example, ¹²⁵I, ¹³¹I, ³²P, ³H, ³⁵S and ¹⁴C), fluorescent compounds (such as, for example, fluorescein, rhodamine, ruthenium tris bipyridyl and lanthanide chelate derivatives), chemiluminescent compounds (such as, for example, acridinium and luminol), visible or fluorescent particles, nucleic acids, complexing agents, and enzymes (such as, for example, alkaline phosphatase, acid phosphatase, horseradish peroxidase, β-galactosidase, β-lactamase and luciferase). In the case of enzyme use, addition of, for example, a chromo-, fluoro-, or lumogenic substrate preferably results in generation of a detectable signal. Other detection systems such as time-resolved fluorescence, internal-reflection fluorescence, and Raman spectroscopy optionally are also useful.

The present invention also desirably provides for the use of detectable labels that are detected indirectly. Indirectly detectable labels typically involve the use of an “affinity pair” i.e. two different molecules, where a first member of the pair is coupled to the detection peptide of the present invention, and the second member of the pair specifically binds to the first member. Binding between the two members of the pair is typically chemical or physical in nature. Examples of such binding pairs include, but are not limited to: antigens and antibodies; avidin/streptavidin and biotin; haptens and antibodies specific for haptens; complementary nucleotide sequences; enzyme cofactors/substrates and enzymes; and the like.

In one embodiment of the present invention, preferably the conjugate moiety is a detectable label. In another embodiment, preferably the detectable label is an enzyme. In a further embodiment, preferably the detectable label is horseradish peroxidase (HRP) or alkaline phosphatase.

The present invention also contemplates that the conjugate moiety is a solid support or particle, or a moiety that facilitates immobilization on a solid support. Examples of suitable solid supports and particles include porous and non-porous materials, latex particles, magnetic particles, microparticles (see, for example, U.S. Pat. No. 5,705,330), beads, membranes, microtiter wells and plastic tubes. Examples of moieties that facilitate immobilization on a solid support include bovine serum albumin (BSA), casein, and thyroglobulin.

### Thiol-Reactive Functionality

A number of different thiol-reactive functionalities known in the art are suitable for use as the sulfhydryl reactive reagent in the process of the present invention. Examples include, but are not limited to, iodoacetamides, maleimides, benzyl halides and bromomethyl ketones. It is within the knowledge of a worker skilled in the art to select an appropriate thiol-reactive functionality based on the particular antigenic protein and conjugate moiety being employed and the downstream application of the resulting protein conjugate.

In one embodiment of the present invention, preferably the thiol-reactive functionality included in the sulfhydryl reactive reagent is a maleimide.

### Linker

In one embodiment of the present invention, the conjugate moiety preferably is linked to the thiol-reactive group via a linker. Linkers of various lengths and conformations as known in the art may be used provided that they do not interfere with the function of the resulting protein conjugate. The linker preferably can comprise one or more functional groups that are stable to the reducing conditions of the present invention. For example, such functional groups preferably include alkyl, cycloalkyl, ether, peptides, or combinations thereof.

In accordance with one embodiment of the present invention, preferably the linker is a branched or unbranched, saturated or unsaturated, hydrocarbon chain having from about 1 to about 100 carbon atoms, optionally from about 1 to about 50 carbon atoms, wherein one or more of the carbon atoms is optionally replaced by —O—, —NR— (wherein R is H, or C1 to C6 alkyl), cycloalkyl group, aryl group, heterocyclic group or heteroaryl group, and wherein the chain is optionally substituted on carbon with one or more substituents selected from the group of (C1-C6) alkoxy, (C3-C6) cycloalkyl, (C1-C6) alkanoyl, (C1-C6) alkanoyloxy, (C1-C6) alkoxycarbonyl, (C1-C6) alkylthio, amide, azido, cyano, nitro, halo, hydroxy, oxo (═O), carboxy, aryl, aryloxy, heteroaryl, and heteroaryloxy.

Examples of suitable linkers include, but are not limited to: peptides preferably having a chain length of from 1 to about 100 atoms, optionally from about 1 to about 50 atoms; linkers preferably derived from groups such as ethanolamine, ethylene glycol and polyethylene, optionally with a chain length of from about 6 to about 100 carbon atoms, optionally from about to about 50 carbon atoms; phenoxyethanol; propanolamide; butylene glycol; butyleneglycolamide; propyl phenyl; and ethyl, propyl, hexyl, steryl, cetyl, and palmitoyl alkyl chains. Other examples preferably include linkers based on polyethylene glycol with from about 3 to about 30 repeating units.

In one embodiment, preferably the linker is a branched or unbranched, saturated or unsaturated, hydrocarbon chain, optionally having from 1 to about 50 carbon atoms, wherein one or more of the carbon atoms is optionally replaced by —O— or —NR— (wherein R is as defined above), and wherein the chain is optionally substituted on carbon with one or more substituents selected from the group of (C1-C6) alkoxy, (C1-C6) alkanoyl, (C1-C6) alkanoyloxy, (C1-C6) alkoxycarbonyl, (C1-C6) alkylthio, amide, hydroxy, oxo (═O), carboxy, aryl, and aryloxy.

In another embodiment, preferably the linker is a branched or unbranched, saturated or unsaturated, hydrocarbon chain, optionally having from 1 to about 50 carbon atoms, wherein one or more of the carbon atoms is optionally replaced by a cycloalkyl group or an aryl group. In a further embodiment, preferably the linker comprises a cyclohexyl group or a phenyl group. In another embodiment, preferably the linker additionally comprises one or more amide bonds.

In another embodiment, preferably the linker is an unbranched, saturated hydrocarbon chain, optionally having from 1 to about 50 carbon atoms, wherein one or more of the carbon atoms is optionally replaced by —O— or —NR— (wherein R is as defined above), and wherein the chain is optionally substituted on carbon with one or more substituents selected from the group of (C1-C6) alkoxy, (C1-C6) alkanoyl, (C1-C6) alkanoyloxy, (C1-C6) alkoxycarbonyl, (C1-C6) alkylthio, amide, hydroxy, oxo (═O), carboxy, aryl and aryloxy.

Non-limiting examples of commercially available heterobifunctional cross-linking reagents having activated esters which can react with primary amines on target molecules, and maleimides and iodoacetamides thiol-reactive functionalities include N-succinimidyl 4-(maleimidomethyl)cyclohexanecarboxylate; 3-(maleimido)propionic acid N-hydroxysuccinimide ester; 11-(maleimido)undecanoic acid N-succinimidyl ester; maleimidoacetic acid N-hydroxysuccinimide ester; 3-maleimidobenzoic acid N-hydroxysuccinimide ester; 4-maleimidobutyric acid N-succinimidyl ester; 6-maleimidohexanoic acid N-succinimidyl ester; 4-(4-maleimidophenyl)butyric acid N-succinimidyl ester; 3-(maleimido)propionic acid N-succinimidyl ester; succinimidyl-4-iodoacetyl-aminobenzoate and 6-(iodoacetamido)hexanoic acid N-hydroxysuccinimide ester.

### Reaction Conditions

The reduction and conjugation steps which optimally comprise the in situ process of one embodiment of the invention are set forth schematically in FIG. 1. The reduction step in which the antigenic protein is contacted with the trialkylphosphine reducing agent preferably can be conducted under a range of reaction conditions as known in the art (see, for example, Han, J. C., et al., Anal. Biochem., 220, 5-10 (1994); Mery, J. C., et al., Int. J. Peptide Protein Res., 42, 44-52 (1993); Ruegg, U. T. and Rudinger, J., Methods Enzymol., 47, 111-126 (1997)). Suitable reaction conditions are also as provided by the manufacturers of commercially available trialkylphosphines.

For example, TCEP or other trialkylphosphine preferably is used at from a 1- to about a 100-fold molar excess over protein concentration. In one embodiment of the present invention, TCEP preferably is used as the reducing agent at from a 1- to about a 75-fold molar excess over protein concentration. In another embodiment, TCEP preferably is used as the reducing agent at from a 1- to about a 50-fold molar excess over protein concentration. In a further embodiment, TCEP preferably is used as the reducing agent at from a 1- to about a 20-fold molar excess over protein concentration. In still another embodiment, TCEP preferably is used as the reducing agent at from a 2- to about a 20-fold molar excess over protein concentration.

The reaction preferably can be conducted over a range of temperatures between about 5° C. and about 100° C., and optionally at a pH between about pH 2 and about pH 11. The optimal length of time for the reaction will be dependent on the temperature at which the reaction is conducted. Thus, for low temperatures, the reaction time may be in the order of hours, whereas at room temperature or above, the reaction is generally complete after from about 5 minutes to about three hours. By way of example, protein reduction preferably can be achieved within about 30 minutes using from about 2 mM to about 3 mM of TCEP for aqueous protein solutions of from about 2 mg to about 10 mg. Appropriate reaction conditions can be readily determined by one skilled in the art.

In one embodiment, preferably the reduction step is conducted in the presence of a molar excess of trialkylphosphine at a temperature of about 100° C. for between about 8 minutes and about 15 minutes. In an alternative embodiment, preferably the reduction step is conducted in the presence of a molar excess of trialkylphosphine at room temperature for between about 30 minutes and about 3 hours.

As noted above, the trialkylphosphine preferably need not be removed from the reaction mixture prior to the conjugation step. Optionally, as described above, other components can be employed in the reduction and/or conjugation step. For example, SDS may be employed with TPEP or other triaklyphosphine, preferably from about a 1- to about a 10-fold molar excess of SDS over protein can be used. In one embodiment of the present invention, preferably from about a 1- to about a 15-fold molar excess of SDS over protein is used. In another embodiment, preferably about a 2- to about a 8-fold molar excess of SDS over protein is used. In yet another embodiment, preferably about a 5- to about a 7-fold molar excess of SDS over protein is used.

The conjugation step also preferably can be conducted under a range of conditions depending on the particular protein and sulfhydryl reactive reagent employed. Appropriate conditions can be readily determined by the skilled technician.

For example, for sulfhydryl reactive reagents comprising iodoacetamide, from about a 1- to about a 20-fold molar excess of sulfhydryl reactive reagent over protein, optionally from about a 1- to about a 10-fold molar excess of sulfhydryl reactive reagent over protein, can be used. In one embodiment of the present invention, preferably for sulfhydryl reactive reagents comprising iodoacetamide, from about a 1- to about a 15-fold molar excess of sulfhydryl reactive reagent over protein is used. In another embodiment, preferably for sulfhydryl reactive reagents comprising iodoacetamide, from about a 2- to about a 5-fold molar excess of sulfhydryl reactive reagent over sulfhydryl content is used. For iodoacetamide reagents, the conjugation reaction preferably should be carried out in the absence of light.

For sulfhydryl reactive reagents comprising iodoacetamide, preferably the conjugation step is conducted at a pH from between about 6.0 and about 9.0 and a temperature between about 2° C. and about 40° C. In one embodiment of the present invention, for sulfhydryl reactive reagents comprising iodoacetamide, preferably the reaction is carried out at a pH between about 6.0 to about 8.5. In another embodiment of the present invention, for sulfhydryl reactive reagents comprising iodoacetamide, preferably the reaction is carried out at a pH between about 7.5 to about 8.5.

For sulfhydryl reactive reagents comprising iodoacetamide, reaction times between about 15 minutes and about 24 hours are preferable depending on the reaction temperature employed. For example, at low temperatures of between about 2° C. and about 10° C., reaction times of between about 2 hours and about 24 hours are preferable. At elevated temperatures, for example between room temperature and about 40° C., reaction times of between about 30 minutes and about 4 hours are preferable.

As a further example, for sulfhydryl reactive reagents comprising maleimide, preferably about from a 1- to about a 20-fold molar excess of sulfhydryl reactive reagent over protein can be used. In one embodiment of the present invention, for sulfhydryl reactive reagents comprising maleimide, preferably from about a 1- to about a 15-fold molar excess of sulfhydryl reactive reagent over protein is used. In another embodiment, for sulfhydryl reactive reagents comprising maleimide, preferably about from a 1- to about a 10-fold molar excess of sulfhydryl reactive reagent over protein is used.

For sulfhydryl reactive reagents comprising maleimide, preferably the conjugation step is conducted at a pH from between about 6.0 to about 8.0, and at a temperature from between about 2° C. to about 40° C. Reaction times between about 30 minutes and about 24 hours are preferable depending on the reaction temperature employed. For example, at low temperatures from between about 2° C. to about 10° C., reaction times from between about 2 hours to about 24 hours are preferable. At elevated temperatures, for example from between room temperature to about 40° C., reaction times of from between about 30 minutes to about 4 hours are preferable. Appropriate conditions can be readily determined by the skilled technician.

In one embodiment of the present invention, preferably the process employs a molar excess of a sulfhydryl reactive reagent comprising a maleimide, and preferably the conjugation step is conducted at a temperature between about 30° C. and about 40° C. for a reaction time between about 30 minutes and about 2 hours. In an alternative embodiment of the invention, preferably the process employs a molar excess of a sulfhydryl reactive reagent comprising a maleimide, and preferably the conjugation step is conducted at a temperature between about 2° C. and about 8° C., preferably for a reaction time between about 2 hours and about 24 hours.

### Additional Steps

The process may optionally further comprise one or more additional steps subsequent to the conjugation step. For example, preferably the process can include a blocking step, a quenching step, a desalting step, one or more purification steps, or combinations thereof.

In one embodiment, blocking or quenching steps can be employed to prevent further reaction of any free sulfhydryl groups remaining unreacted after the conjugation step. Blocking can be achieved as is known in the art, for example, through the use of iodoacetic acid, maleimide derivatives (such as, for example, N-ethylmaleimide), cysteine, iodoacetamide, and various salts of iodoacetic acid. Quenching preferably can be achieved through addition of a suitable reducing agent, such as, for example, cysteine, DTT, TCEP, β-mercaptoethanol and the like.

Desalting preferably can be achieved through the use of an appropriate desalting column (such as, for example, those commercially available from GE Healthcare Bio-Sciences AB, Uppsala, Sweden, and Pierce Biotechnology Inc., Rockford, Ill.), or by dialysis.

Preferably various purification procedures may also be employed, including, but not limited to, chromatography-based steps, such as gel filtration (e.g., using a PD10 column), size exclusion, ion-exchange and the like. Additional purification procedures including, but not limited to, dialysis, filtration, and tangential flow filtration can also be employed.

In one embodiment of the invention, preferably a reduction and conjugation step is followed by a blocking step, for example, blocking with iodoacetic acid, for instance at about 3 hours at room temperature. Other variations easily can be determined by one skilled in the art.

### Characteristics of the Antigenic Protein Conjugate

In accordance with one embodiment of the present invention, preferably the antigenic protein conjugates provided by the process of the present invention demonstrate enhanced antibody-binding properties when compared to the corresponding sulfhydryl derivatized conjugate prepared by other methods, such as those employing DTT or β-mercaptoethanol, and thus represents an improvement over currently available sulfhydryl derivatized protein conjugates.

Enhanced antibody binding can be demonstrated by, for example, increased sensitivity in an immunoassay format. Increased sensitivity can be, for example, the ability to detect lower titers of antibodies and/or the ability to detect the same titer of antibodies when used at a lower concentration. In one embodiment, the antigenic protein conjugate produced by the in situ method preferably can be employed in an immunoassay at lower concentrations to detect substantially equivalent titers of antibody when compared to a corresponding sulfhydryl derivatized conjugate prepared by conventional methods (i.e., a “conventional” sulfhydryl derivatized conjugate) under the same assay conditions.

In many immunoassay situations, protein conjugates that are derivatized through sulfhydryl groups require the presence of a reducing agent in the assay in order to bind effectively to their cognate antibody or antibodies. By contrast, in one embodiment of the present invention, the antigenic protein conjugate prepared by the in situ process can efficiently bind to cognate antibodies without requiring the presence of a reducing agent.

### Applications

The antigenic protein conjugates prepared by the process of the present invention have application in a number of contexts. For example, the protein conjugates preferably can be used in applications where detection of the cognate antibody or antibodies is required, such as diagnostic assays where the conjugate moiety is a detectable label, as well as optionally in applications where purification of the cognate antibody or antibodies is required, for example as an affinity ligand wherein the conjugate moiety is a solid support or particle, or a moiety that facilitates immobilization on a solid support. The present invention also provides for the use of the antigenic protein conjugates as research tools, for example, in the development of assays, or in the isolation of antibodies to a particular target protein.

In a further aspect, the present invention thus provides a method of detecting an antibody to an antigenic protein preferably employing an antigenic protein conjugate prepared by the in situ process. In one embodiment, the protein conjugates preferably are employed in diagnostic immunoassays.

In another aspect, the present invention also provides for immunoassay kits preferably comprising one or more antigenic protein conjugate. The antigenic protein conjugate preferably can be provided in the kit as a capture antigen wherein the conjugate moiety is a solid support or particle, and/or as a detection antigen wherein the conjugate moiety is a detectable label.

In a specific embodiment, the protein conjugate is a HCV NS3 conjugate which preferably is employed in a combination HCV antibody-antigen detection assay. Of course, it goes without saying that any of the exemplary formats herein, and any assay or kit according to the invention, can be adapted or optimized for use in automated and semi-automated systems (including those in which there is a solid phase comprising a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM, IMX, PRISM, and Quantum II platforms, as well as other platforms.

Additionally, the assays and kits of the present invention optionally can be adapted or optimized for point of care assay systems, including Abbott's Point of Care (1-STAT™) electrochemical immunoassay system. Immunosensors and methods of manufacturing and operating them in single-use test devices are described, for example in U.S. Pat. No. 5,063,081 and published US Patent Applications 20030170881, 20040018577, 20050054078, and 20060160164 (incorporated by reference herein for their teachings regarding same).

The invention will now be described with reference to specific examples. It will be understood that the following examples are intended to describe illustrative embodiments of the invention and are not intended to limit the invention in any way.

### EXAMPLES

### Example 1

### In Situ Preparation of a Conjugate of Hepatitis C Virus NS3 Protein and Horseradish Peroxidase

A conjugate of recombinant hepatitis C virus (HCV) non-structural protein NS3 and horseradish peroxidase (HRP) was prepared as follows. The recombinant NS3 (rNS3) was prepared according to standard protein expression methods and comprised the native sequence of NS3 together with a leader sequence from the vector at a position N-terminal to the native sequence.

An HRP-maleimide solution was prepared as follows. A 2-fold molar excess of sulfo-SMCC was dissolved in DMSO (Pierce) and added to 100 mg/mL HRP dissolved in 25 mM HEPES/1 mM EDTA, pH 7.8. The solution was swirled gently and left for 45 minutes at room temperature. The HRP-maleimide was purified by gel filtration by loading the solution (2.5 mL) on a PD10 column (Pharmacia) and eluting with 25 mM HEPES/1 mM EDTA, pH 6.8 (3.2 mL).

The rNS3 was reduced in an aqueous solution of TCEP (Perbio or Calbiochem) containing a 10-fold excess of TCEP, at a pH of 6.8 for 2 hours at room temperature to provide the reduced rNS3. Ten molar equivalents of horseradish peroxidase (HRP)-maleimide, prepared as described above, were added to the solution of reduced rNS3 and TCEP, and the resulting mixture was swirled and left to stand for 16-24 hours at 2-8° C. to produce rNS3-HRP. Any unreacted sulfhydryl groups were then blocked by reaction with an excess of iodoacetic acid for 3 hours at room temperature. The resulting blocked rNS3-HRP was purified by gel filtration on a PD10 column (Sephadex™ G-25 Medium; GE Healthcare Bio-Sciences AB, Uppsala, Sweden) by loading 2.5 mL of product onto each column and eluting in 3 mL provide the final rNS3-HRP product. The total time required to carry out the four-step procedure to prepare the final rNS3-HRP product was 1½ days.

### Example 2

### Preparation of a Conjugate of Hepatitis C Virus NS3 Protein and Horseradish Peroxidase by Conventional Techniques

In contrast to the in situ process described in Example 1, the most efficient prior method for preparation of rNS3-HRP conjugate required nine steps (including purification of intermediate products) and took a total time of 3 days.

Specifically, rNS3 was incubated with a final concentration of approximately 300 mM β-mercaptoethanol (β-ME). The reduced rNS3 was then loaded on a G25 Sephadex column, and eluted with 8 M urea/EDTA/25 mM HEPES, pH 7.8. Eluted fractions were tested for the presence of protein and fractions containing the protein were then pooled. The pooled rNS3 fractions were then incubated with a 50-fold molar excess of SATA for 1 hour at 30° C. The solution was dialyzed twice in 6 M urea/EDTA/50 mM HEPES, pH 6.8, at 2-8° C., first for 6 hours, then overnight. Incorporation of thiol groups into the protein was analyzed by standard methods.

The reduced rNS3 was incubated with a 13-fold molar excess of HRP-maleimide overnight at 2-8° C. and demasking was achieved simultaneously by addition of hydroxylamine at an approximately 130-fold molar excess over the protein. β-ME was then added as a 30-fold molar excess over the protein and incubated for 20 minutes at 2-8° C. NEM (N-ethyl maleimide) was then added as 100-fold excess over the protein, resulting in the rNS3-HRP conjugate. Some of the conventional HCV conjugates may have been prepared with these last two steps (blocking with β-ME, and NEM addition) omitted.

### Example 3

### Comparison of the Sensitivity of NS3 Protein Conjugates Prepared by In Situ and Conventional Techniques

The sensitivity of the rNS3-HRP conjugates prepared by the in situ process as described in Example 1 and by the conventional technique as described in Example 2 was compared by assessing the ability of each conjugate to detect antibodies to HCV using a positive HCV serum sample (QC1691) at various dilutions of conjugate, and determining the level of background with HCV negative samples, using an HCV immunoassay kit comprising immobilized NS3 protein. This kit is the subject of a patent application entitled “Combination Hepatitis C Virus Antigen and Antibody Detection Method”, filed Sep. 1, 2006 as U.S. Patent Application No. 60/841,800 (incorporated by reference for its teachings regarding same). The conjugates prepared in Examples 1 and 2 were substituted for the conjugates that are normally provided with the immunoassay kit.

The immunoassay kit included the following components:

1. Each plate of 96 wells was coated with purified recombinant HCV antigen (rNS3), core protein peptides and anti-HCV core monoclonal antibody.

2. Sample diluent having the chemical composition shown in Table 1 and a pH of 6.2.

TABLE 1
Chemical Composition of the Sample Diluent

| CAS Number | Chemical/Biological Substance Name | Wt/Wt % |
|---|---|---|
| 7365-45-9 | HEPES | 0.220% |
| 9002-93-1 | Triton-X-100 | 3.925% |
| 3458-28-4 | Mannose | 9.259% |
| 50-01-1 | Guanidine | 3.537% |
| 57-13-6 | Urea | 2.224% |
| 7647-14-5 | Sodium Chloride | 2.164% |
| 115-40-2 | Bromocresol Purple (Sodium Salt) | 0.018% |
| 910010-01-6 | Filtered Heated Calf Serum | 4.630% |
| 1186 | Sodium Alkyl Paraben (Nipasept)* | 0.317% |
| 990001-12-8 | A56620 | 0.925% |
| 910010-14-0 | HB05 Post Protein G | 0.557% |
| 6381-92-6 | EDTA | 1.850% |
| 7732-18-5 | Distilled water | 70.414% |

*The sodium alkyl paraben contains: sodium 4-(methoxycarbonyl) phenolate (CAS No. 5026-62-0; 0.199%), sodium 4-ethoxycarbonylphenoxide (CAS No. 35285-68-8; 0.047%) and sodium 4-propoxycarbonylphenoxide (CAS No. 35285-69-9; 0.030%).

3. Negative control having the chemical composition shown in Table 2 and a pH of 7.6.

TABLE 2
Chemical Composition of Negative Control Solution

| CAS Number | Chemical/Biological Substance Name | Wt/Wt % |
|---|---|---|
| 7365-45-9 | HEPES | 1.260% |
| 910010-01-5 | 10% casein | 10.582% |
| 910010-06-2 | Base Matrix | 52.910% |
| 990001-12-8 | A56620 | 1.058% |
| 1186 | Sodium Alkyl Paraben (Nipasept)* | 0.317% |
| 3844-45-9 | Blue Dye | Trace |
| 910010-13-8 | Tartrazine | Trace |
| 7732-18-5 | Distilled Water | 33.871% |

*The sodium alkyl paraben contains: sodium 4-(methoxycarbonyl) phenolate (CAS No. 5026-62-0; 0.228%), sodium 4-ethoxycarbonylphenoxide (CAS No. 35285-68-8; 0.054%) and sodium 4-propoxycarbonylphenoxide (CAS No. 35285-69-9; 0.035%).

4. Antibody positive control having the chemical composition shown in Table 3 and a pH of 7.6.

TABLE 3
Chemical Composition of Antibody Positive control

| CAS Number | Chemical/Biological Substance Name | Wt/Wt % |
|---|---|---|
| 7365-45-9 | HEPES | 1.260% |
| 910010-01-5 | 10% Casein | 10.580% |
| 910010-06-2 | Base Matrix | 52.900% |
| 990001-12-8 | A56620 | 1.058% |
| 1186 | Sodium Alkyl Paraben (Nipasept)* | 0.317% |
| 910010-017 | Holly Red Dye | Trace |
| 910010-13-8 | Tartrazine | Trace |
| 990001-03-0 | HCV Positive Sera | 0.755% |
| 7732-18-5 | Distilled Water | 33.128% |

*The sodium alkyl paraben contains: sodium 4-(methoxycarbonyl) phenolate (CAS No. 5026-62-0; 0.228%), sodium 4-ethoxycarbonylphenoxide (CAS No. 35285-68-8; 0.054%) and sodium 4-propoxycarbonylphenoxide (CAS No. 35285-69-9; 0.035%).

5. Antigen positive control having the chemical composition of shown in Table 4 and a pH of 10.8 to 11.2.

TABLE 4
Chemical Composition of the Antigen Positive Control Solution

| CAS Number | Chemical/Biological Substance Name | Wt/Wt % |
|---|---|---|
| 7647-14-5 | Sodium Chloride | 0.877% |
| 910010-01-5 | 10% Casein | 1.000% |
| 9005-64-5 | Tween 20 | 0.400% |
| 910010-017 | Holly Red Dye | 0.020% |
| 26628-22-8 | Sodium Azide | 0.008% |
| 1336-21-6 | Ammonium Hydroxide | 0.300% |
| 990001-12-6 | MBL 408 | Trace |
| 990001-12-7 | MBL 411 | Trace |
| 9048-46-8 | Bovine Serum Albumin 30% | Trace |
| 7732-18-5 | Distilled Water | 97.392% |

6. Conjugate diluent having a chemical composition shown in Table 5 and a pH of 6.8.

TABLE 5
Chemical Composition of Conjugate Diluent

| CAS Number | Chemical/Biological Substance Name | Wt/Wt % |
|---|---|---|
| 7365-45-9 | HEPES | 0.424% |
| 7647-14-5 | Sodium Chloride | 4.405% |
| 14933-09-6 | Zwittergent | 0.070% |
| 9002-93-1 | Triton X-100 | 0.142% |
| 9005-64-5 | Tween 20 | 0.072% |
| 8047-15-2 | Saponin | 2.122% |
| 151-21-3 | Sodium Dodecyl Sulfate (SDS) | 0.036% |
| 466 | Proclin 300 | 0.094% |
| 9048-46-8 | 30% BSA | 28.300% |
| 910010-13-4 | Succinylated Casein | 7.800% |
| 910010-01-5 | 10% Casein | 1.349% |
| 121-79-9 | N-Propyl Gallate | TRACE |
| 64-17-5 | Absolute Alcohol | 0.030% |
| 9003-99-0 | Horse Radish Peroxidase | 1.000% |
| 7732-18-5 | Distilled Water | 54.155% |

8. Substrate diluent containing 0.048% hydrogen peroxide solution, 4.233% tri-sodium citrate, and 95.719% distilled water and having a pH of 7.5 to 8.5. All percentages are calculated as weight/weight percentages.

9. Substrate concentrate having a chemical composition of the substrate concentrate is shown in Table 6 and a pH of 2.0±0.3.

TABLE 6
Chemical Composition of the Substrate Concentrate

| CAS Number | Chemical/Biological Substance Name | Wt/Wt % |
|---|---|---|
| 64285-73-0 | 3,3′,5,5′-tetramethylbenzidine | 0.038% |
| | dihydro-chloride | |
| 5949-29-1 | Citric Acid (monohydrate) | 4.39% |
| 25100-12-3 | N-Cyclohexylhydroxylamine hydrochloride | 0.014% |
| 6381-92-6 | Ethylenediaminetetra tetra acetic acid | 0.002% |
| | disodium salt dehydrate | |
| 6132-04-3 | Tri-sodium Citrate | 0.01% |
| 62625-31-4 | m-Cresol purple sodium salt | 0.002% |
| 7732-18-5 | Water (distilled) | 95.544% |

### Substrate Solution

To prepare the Substrate Solution a volume of colorless Substrate Diluent is added to an equal volume of orange Substrate Concentrate, as indicated in Table 7 below, in a clean plastic vessel.

TABLE 7
Volume of Substrate Concentrate and Substrate Diluent required

| Number of Wells | | | | | | | | | Number of Plates | | | | | |
|---|---|---|---|---|---|---|---|---|---|---|---|---|---|---|
| 8 | 16 | 24 | 32 | 40 | 48 | 56 | 64 | 72 | 80 | 96 | 1 | 2 | 3 | 4 |

Substrate Concentrate (mL)

| | | | | | | | | | | | | | | |
|---|---|---|---|---|---|---|---|---|---|---|---|---|---|---|
| 1 | 1.5 | 2 | 2.5 | 2.5 | 3 | 3.5 | 4 | 4.5 | 4.5 | 6 | 6 | 12 | 18 | 22 |

Substrate Diluent (mL)

| | | | | | | | | | | | | | | |
|---|---|---|---|---|---|---|---|---|---|---|---|---|---|---|
| 1 | 1.5 | 2 | 2.5 | 2.5 | 3 | 3.5 | 4 | 4.5 | 4.5 | 6 | 6 | 12 | 18 | 22 |

10. The kit includes Wash Fluid. Specifically, one or two bottles containing 125 mL of 20 times working strength Tween/Saline Wash Fluid. The 20 times working strength solution contains 1.714% Bronidox® (10% solution), 1.541% propane-1,2-diol, 0.173% 5-bromo-5-nitro-1,3-dioxane, 14.266% sodium chloride, 0.857% Tween 20, and 83.136% water. All percentages are weight/weight percentages. The pH of the solution is 7, and the density of the solution is 1.11 g/mL. The Wash Fluid is diluted one in twenty with either distilled or deionized water to give the required volume, or the entire contents of one bottle of Wash Fluid is diluted to a final volume of 2500 mL. When diluted, the Wash Fluid contains 0.01% Bronidox® preservative.

### Immunoassay Protocol:

The immunoassay was carried according to the steps described below. All reagents and samples and reagents were allowed to come to 18 to 30° C. before use. Any glassware to be used with the reagents was thoroughly washed with 2 M hydrochloric acid and then rinsed with distilled water or high quality deionized water.

The method comprises the following steps:
1. Prepare the Wash Fluid, reconstitute the Conjugate.
2. Add 50 μl of Sample Diluent to each well.
3. Add 50 μl of Samples or 50 μl of Controls to the wells. The use of a white background will aid the visualization of sample addition.
4. Cover the wells with the lid and incubate for 60 minutes at room temperature (first incubation period).
5. At the end of the incubation period wash the plate as described under Wash Procedures. After washing is complete invert the plate and tap out any residual Wash Fluid onto absorbent paper.
6. Immediately add 100 μl of conjugate to each well.
7. Cover the wells with the lid and incubate for 60 minutes at room temperature (second incubation period).
8. Prepare the substrate solution.
9. Repeat step 5.
10. Immediately after washing the plate, add 100 μl of substrate solution to each well.
11. Cover the wells with a lid and incubate for 30 minutes at 37° C.±1° C. (third incubation period). Keep away from direct sunlight.
12. Add 50 μl of stop solution (0.5M sulfuric acid).
13. Within 15 minutes read the absorbance at 450 nm using 690 nm as the reference wavelength if available. Blank the instrument on air (no plate in the carriage).

Wash Procedures: The plates were washed according to standard protocols as known in the art.

### Results:

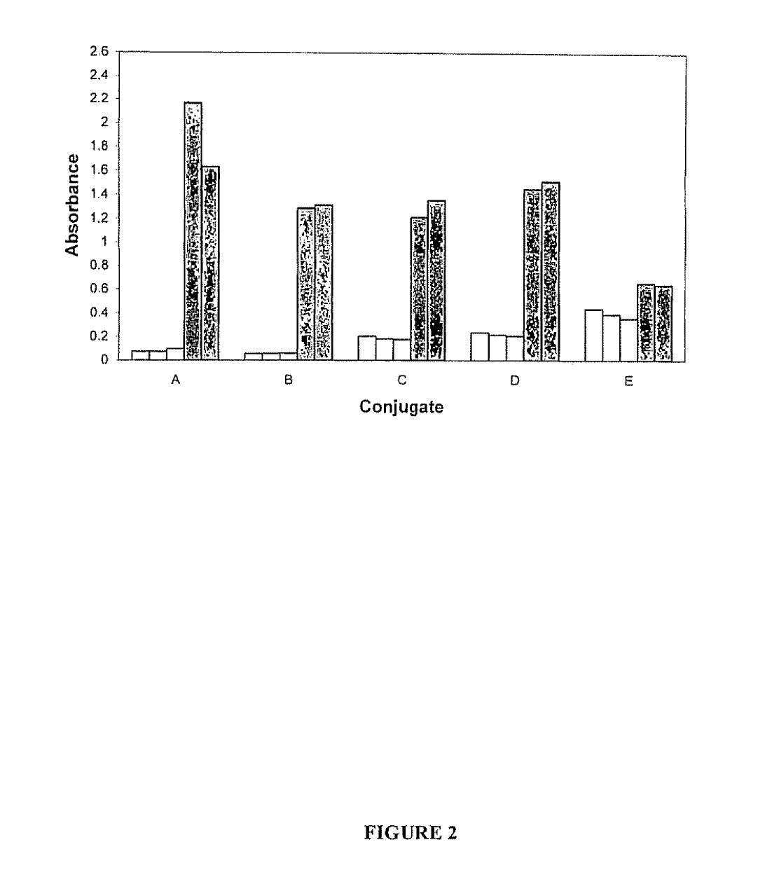

The results of an exemplary experiment are shown in FIG. 2 and indicate that the in situ rNS3-HRP conjugate can be used at a higher titer (i.e. lower concentration) than the conventional rNS3-HRP and gave a higher signal and a lower negative. In addition, the rNS3-HRP conjugate can be used in the absence of DTT or TCEP, whereas for the rNS3-HRP conjugate prepared according to the conventional method, 6 mM DTT had to be added to the conjugate diluent. If the conventional rNS3-HRP was used without DTT or TCEP, the positive signal was substantially diminished. The results suggest that the in situ method holds the protein structure in an open confirmation, and rather than the bulky HRP moiety masking key epitopes, as may be expected, they are revealed in a stable manner.

### Example 4

### In Situ Preparation of a Conjugate of HIV gp41-p24 Recombinant Protein (dx589) and Horseradish Peroxidase Utilizing a Detergent

A conjugate of HIV gp41-p24 recombinant protein (dx589) and horseradish peroxidase (HRP) was prepared as follows. dx589 was reduced with a 5-fold molar excess of TCEP compared to the protein, in an aqueous solution containing 8M urea, 25 mM HEPES, 1 mM EDTA, pH 7.8, and a 6.25 molar excess of sodium dodecyl sulfate (SDS) over the protein for 10 minutes in a heating block at 100° C.

HRP-maleimide was prepared using the protocol as described in Example 1. The reduced dx589 was then reacted with a 5-fold molar excess of HRP-maleimide, for 15-24 hours at 2-8° C.

### Example 5

### In Situ Preparation of a Conjugate of HIV gp41-p24 Recombinant Protein (dx589) and Horseradish Peroxidase Optimization of TCEP Concentration

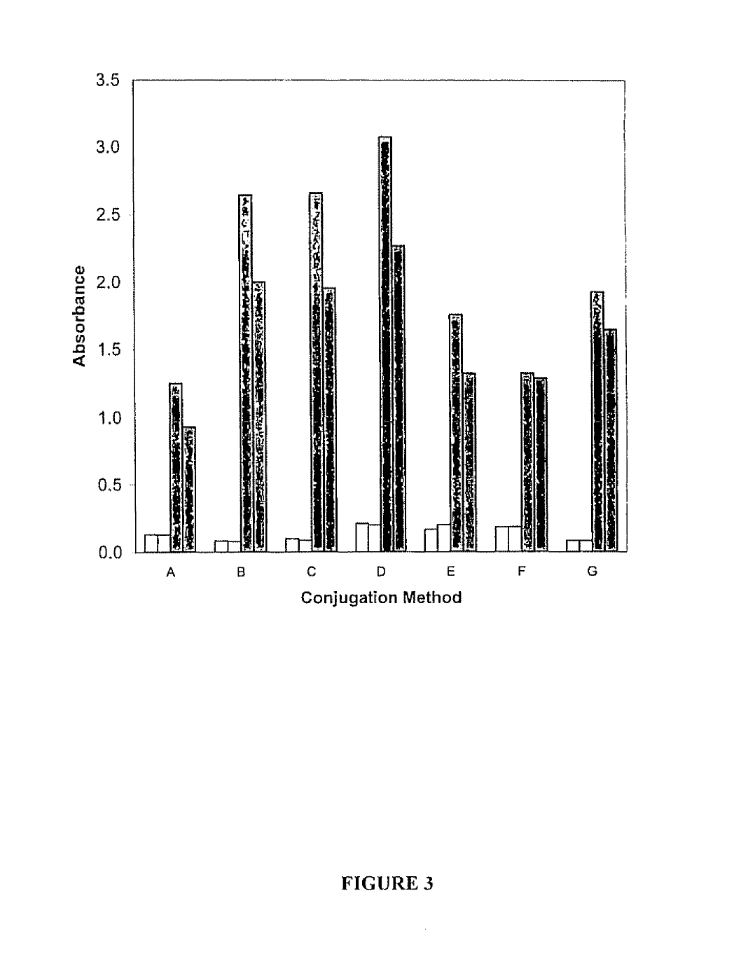

The effect of varying concentration of TCEP in the reduction reaction on the ability of the resulting dx589-HRP conjugate to bind cognate antibodies was investigated. Various dx589-HRP conjugates were prepared as generally described in Example 4, but without the inclusion of SDS in the reduction reaction and employing 5-fold molar equivalents of HRP-maleimide with respect to the protein, and varying concentrations of TCEP (0 to 40-fold molar equivalents with respect to the protein). The conjugates were tested against HIV positive panels QC1362 and QC1363, and results are depicted in FIG. 3.

A dx589-HRP conjugate (“G”, Reference, FIG. 3) was also prepared according to the method described in Example 2 for preparation of the conventional rNS3-HRP conjugate.

The ability of the dx589-HRP conjugate prepared by the in situ method to detect antibodies to HIV using positive HIV serum samples (QC1362 and QC1363, solid bars in FIG. 3) and determination of the level of background with HIV negative samples (open bars in FIG. 3) was assessed using the Abbott Murex HIV 1.2.0 kit, following the protocol supplied with the kit, and compared to dx589-HRP prepared by the conventional technique. The dx589-HRP conjugates were tested by substituting them for the conjugates that are usually supplied with the kit. The results of an exemplary experiment are shown in FIG. 3. Optimal concentrations of TCEP in this context were determined to be between 2.5 and 20-fold molar equivalents. The concentration of TCEP that provided the lowest negative signals with a high positive signal was determined to be 5-fold molar equivalents.

### Example 6

### In Situ Preparation of a Conjugate of HIV gp-41-p24 Recombinant Protein (dx589) and Horseradish Peroxidase Optimization of HRP-Maleimide Concentration

The effect of varying concentration of HRP-maleimide in the conjugation reaction on the ability of the resulting dx589-HRP conjugate to bind cognate antibodies was investigated. Various dx589-HRP conjugates were prepared by the in situ technique as described in Example 5 (i.e., without SDS in the reduction reaction), but employing 5-fold molar equivalents of TCEP and varying concentrations (2.5 to 20-fold molar equivalents) of HRP-maleimide.

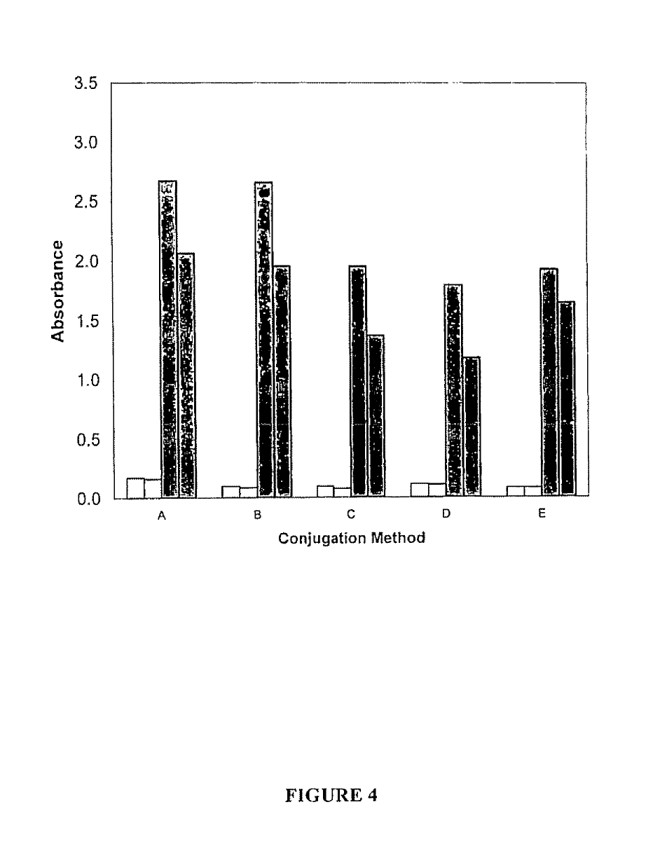

The ability of the resulting dx589-HRP conjugates to detect antibodies to HIV using positive HIV serum samples (QC1362 and QC1363, solid bars in FIG. 4) and determination of the level of background with HIV negative samples (open bars in FIG. 4) was assessed following the procedure generally described in Example 5 and compared to dx589-HRP prepared by the conventional technique (“ref”). The results of an exemplary experiment are shown in FIG. 4. Optimal concentrations of HRP-maleimide for this purpose were determined to be between 2.5- and 10-fold molar equivalents. The concentration of HRP-maleimide that provided the lowest negative signals with a high positive signal was determined to be a 5-fold molar excess.

### Example 7

### Titration of dx589-HRP Conjugates Prepared by an In Situ Technique

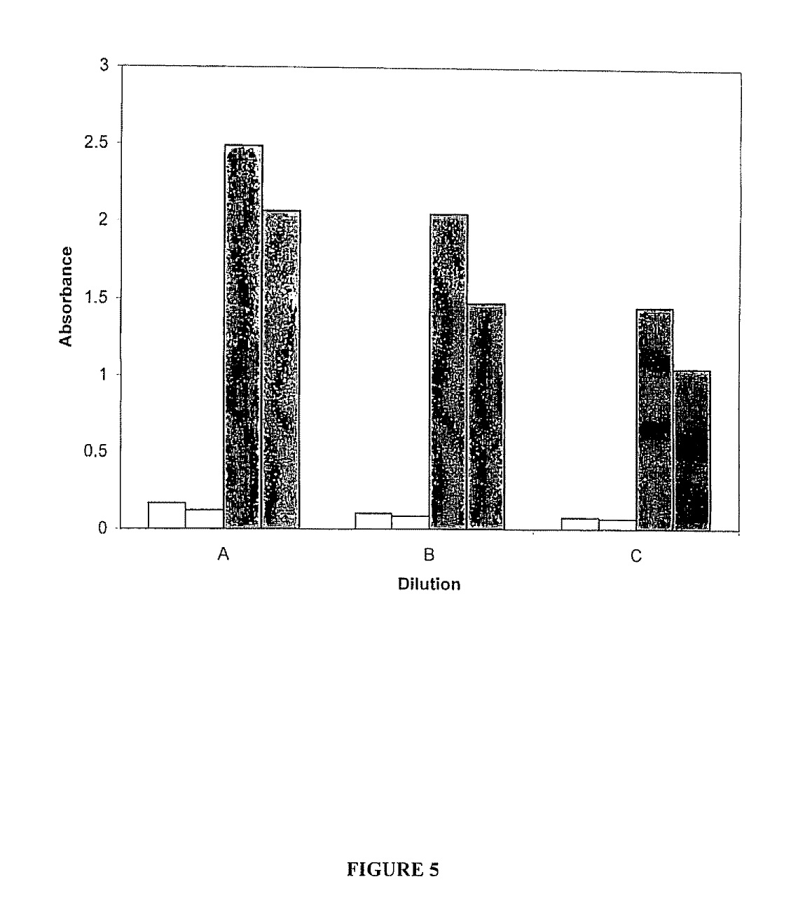

The sensitivity of the dx589-HRP conjugate prepared as described in Example 4 in binding to antibodies in HIV positive samples was assessed by serially diluting a preparation of the dx589-HRP conjugate and determining the ability of the resulting diluted dx589-HRP conjugates to detect antibodies to HIV using positive HIV serum samples (QC1362 and QC1363, solid bars in FIG. 5) and the level of background observed with HIV negative samples (open bars in FIG. 5) following the procedure generally described in Example 5. The results of an exemplary experiment are shown in FIG. 5 and demonstrate that the dx589-HRP conjugate prepared by the in situ process was able to specifically detect antibodies even at high dilutions (1 in 52,000).

### Example 8

### Comparison of the Specificity of dx589 Protein Conjugates Prepared by In Situ Techniques and Conventional Techniques

The specificity of dx589-HRP conjugates prepared by the in situ methods described in Example 5 (no SDS) and Example 4 (+SDS) was assessed against a panel of HIV negative samples following the assay procedure described in Example 5 and the number of false positives determined and compared to the number of false positives obtained with the same panel of samples using dx589-HRP prepared by the conventional technique.

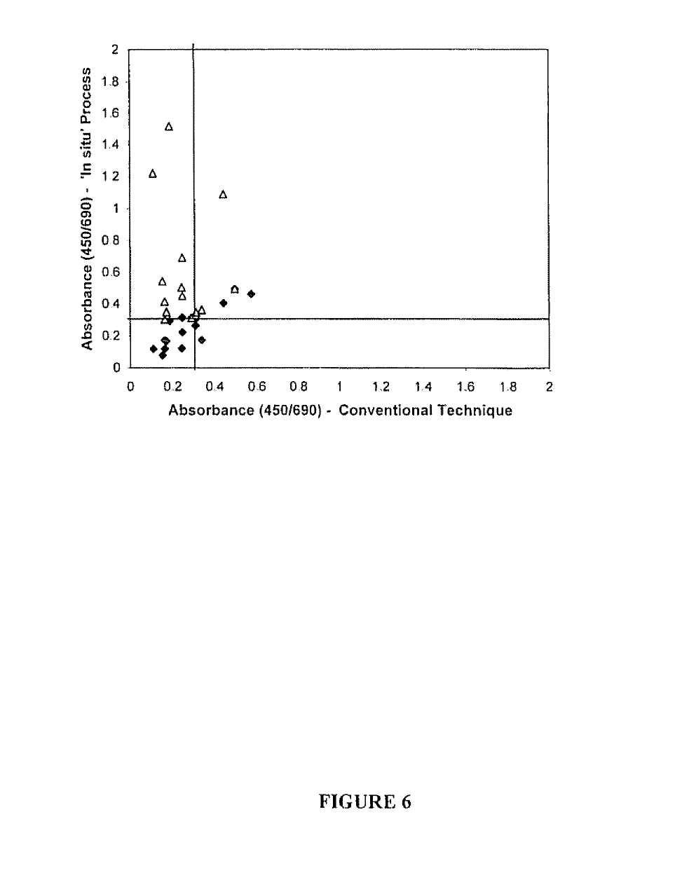

The results of an exemplary experiment are shown in FIG. 6. The open triangles present the comparison data for dx589-HRP conjugate prepared by the in situ process (no SDS) vs. dx589-HRP prepared by the conventional technique. The solid diamonds present the comparison data for dx589-HRP conjugate prepared by the in situ process (+SDS) vs. dx589-HRP prepared by the conventional technique. The horizontal and vertical lines depict “cut off” values. Values above the cut off are considered to be “false positives.” The results demonstrate that the dx589-HRP conjugate prepared by the in situ process (+SDS) has a specificity equal to the dx589-HRP prepared by the conventional technique. The specificity of the dx589-HRP conjugate prepared by the in situ process (no SDS) in these experiments was less than satisfactory. In addition, the dx589-HRP conjugate prepared by the in situ process could be prepared more consistently and gave more reproducible results than the conjugate prepared by conventional techniques.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

Although the invention has been described with reference to certain specific embodiments, various modifications thereof will be apparent to those skilled in the art without departing from the spirit and scope of the invention as outlined in the claims appended hereto.

What is claimed is:

1. A process for preparing an antigenic protein conjugate comprising the steps of:
(a) contacting an antigenic protein comprising one or more disulfide bonds with a trialkylphosphine under conditions that allow reduction of at least one of said one or more disulfide bonds to provide a reduced antigenic protein comprising at least a pair of sulfhydryl residues, and
(b) contacting said reduced antigenic protein with a sulfhydryl reactive reagent comprising a conjugate moiety and a thiol-reactive functionality under conditions that allow reaction of at least one of said sulfhydryl residues with the sulfhydryl reactive reagent to form said antigenic protein conjugate.

2. The process of claim 1, wherein said trialkylphosphine is selected from the group consisting of tris(2-carboxyethyl)phosphine, tris(2-cyanoethyl)phosphine and tributylphosphine.

3. The process of claim 1, wherein said antigenic protein is a viral protein.

4. The process of claim 1, wherein said antigenic protein is a hepatitis C virus protein or a human immunodeficiency virus protein.

5. The process of claim 1, wherein said antigenic protein is a hepatitis C virus protein.

6. The process of claim 5, wherein said hepatitis C virus protein is non-structural protein NS3.

7. The process of claim 1, wherein said conjugate moiety is a detectable label.

8. The process of claim 1, wherein said conjugate moiety is an enzyme.

9. The process of claim 8, wherein said enzyme is horseradish peroxidase.

10. The process of claim 1, wherein said thiol-reactive functionality is a maleimide or an iodoacetamide.

11. The process of claim 1, wherein said thiol-reactive functionality is a maleimide.

12. An antigenic protein conjugate prepared by a process comprising the steps of:
(a) contacting an antigenic protein comprising one or more disulfide bonds with a trialkylphosphine under conditions that allow reduction of at least one of said one or more disulfide bonds to provide a reduced antigenic protein comprising at least a pair of sulfhydryl residues, and
(b) contacting said reduced antigenic protein with a sulfhydryl reactive reagent comprising a conjugate moiety and a thiol-reactive functionality under conditions that allow reaction of at least one of said sulfhydryl residues with the sulfhydryl reactive reagent to form said antigenic protein conjugate.

13. A method for detecting antibodies to an antigenic protein in a sample comprising:
(i) contacting the sample with an antigenic protein conjugate according to claim 12 under conditions that allow the formation of an antibody:antigenic protein conjugate complex, and
(ii) detecting any antibody:antigenic protein conjugate complex formed in step (i).
